(19) United States
(12) Patent Application Publication
(10) Pub. No.: US 2009/0148506 A1
(43) Pub. Date: Jun. 11, 2009

(54) LIPOSOMAL FORMULATIONS COMPRISING SECONDARY AND TERTIARY AMINES AND METHODS FOR PREPARING THEREOF

(76) Inventors: Awa Dicko, Vancouver (CA); Paul Tardi, Surrey (CA); Lawrence Mayer, North Vancouver (CA); Sharon Johnstone, Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 12/097,515

(22) PCT Filed: Dec. 22, 2006
(86) PCT No.: PCT/US06/49245

§ 371 Date: Oct. 3, 2008

Related U.S. Application Data
(60) Provisional application No. 60/753,644, filed on Dec. 22, 2005.

(51) Int. Cl. A61K 9/127

(52) U.S. Cl. 424/450

(57) ABSTRACT

Provided herein are liposomal compositions comprising a therapeutic agent having a protonatable amino group and a secondary or tertiary amine, and methods for encapsulating such therapeutic agents. In one aspect, the present invention relates to liposomal formulations comprising irinotecan in a triethanolamine solution, and optionally comprising copper gluconate, and methods for preparing the same.

### RELATED APPLICATION

This application claims priority from U.S. application Ser. No. 60/753,644 filed Dec. 22, 2005 which is incorporated herein by reference in its entirety.

### BACKGROUND ART

Two primary techniques are routinely used for the encapsulation of drugs within liposome carriers. One method is passive encapsulation where liposomes are formed in the presence of the drug, See, e.g., Mayer, et al. (1989) Cancer Res. 49: 5922-30. A second, more efficient, “active loading” method involves the formation of transmembrane pH gradients through the use of citrate, ammonium sulfate or ionophore/divalent cation See, e.g., Mayer, et al.(1985) Biochim. Biophys. Acta 813: 294-302; Boman, et al. (1993) Biochim. Biophys. Acta 1152: 253-58; Haran, et al., (1993) Biochim. Biophys. Acta 1151: 201-15; Cullis, et al. (1997) Biochim. Biophys. Acta 1331:187-211; Cheung, et al. (1998) Biochim. Biophys. Acta 1414: 204-16. The acidified liposomal interior causes the loading and retention of drugs with ionizable moieties such as amine groups. See, e.g., Madden et al. (1990) Chem. Phys. Lipids 53: 37-46; Cullis et al. (1991) Tibtech. 9: 57-61. This method allows for efficient drug encapsulation, generally greater than 80%, but also has certain disadvantages. For example, several clinical formulations of such liposomal drugs require the generation of the pH gradient just prior to drug loading due to gradient and/or drug instability. See, e.g., Conley et al. (1993) Cancer Chemother Pharmacol. 33: 107-12; Gelmon et al. (1999) J. Clin. Oncol. 17 (2): 697-705. A second disadvantage is the potential hydrolysis of lipids at acidic pH which can introduce liposome instability during long-term storage. See, e.g., Grit et al. (1993) Chem. Phys. Lipids 64 (1-3): 3-18; Barenholz et al. (1993) Med. Res. Rev. 13 (4): 449-91. Ideally, a loading method would allow for efficient encapsulation at a neutral pH to prevent drug and lipid degradation.

U.S. Pat. Nos. 5,785,987 and 5,800,833 describe methods for loading lipid vesicles using methylammonium ion to create suitable pH gradient for a broad range of loading possibilities. pH gradients between the interior solution and exterior of the liposome allow a drug to cross the liposomal bilayer in the neutral form and then to be trapped within the aqueous interior of the liposome due to conversion of the drug to the charged form in the lower pH interior. Such methods require an internal aqueous solution of very low pH, e.g., pH 4.0, in the liposome while the exterior buffer has a higher pH. However, controlling the pH gradient is critical in maintaining therapeutically useful liposomal compositions. Uncontrolled pH gradients results in drug leakage out of the liposome and/or loss of biological activity as the pH increases in the interior of the liposome. Such liposomes are ineffective and sometimes toxic. These patents also teach the use of ethanolamine or glucosamine as less suitable and inferior gradients for loading a protonatable therapeutic agent. Thus, methods that avoid these problems are advantageous in increasing the effectiveness of liposomes as drug delivery vehicles.

### DISCLOSURE OF THE INVENTION

Provided herein are methods for preparing liposomal compositions containing one or more therapeutic agents in a manner that is independent of pH gradients for loading or encapsulation of the therapeutic agents. The use of a completely neutral system for drug encapsulation facilitates efficient drug loading of the liposomes, preserves the full biological activity of the drug after encapsulation, and increases long term stability of the liposome-encapsulated drugs.

Thus, in one aspect, provided herein is a method of preparing a liposomal composition of at least one therapeutic agent, the method comprising: i) providing a liposomal composition comprising a mixture of liposomes in an aqueous solution, wherein said liposomes have an internal aqueous solution comprising a secondary or tertiary amine aqueous solution, wherein said internal aqueous solution is buffered at a neutral pH; ii) adding a first therapeutic agent to an external aqueous solution, wherein said external aqueous solution is buffered at a neutral pH, and wherein the first therapeutic agent has a protonatable amino group; iii) maintaining the therapeutic agent in the external aqueous solution for sufficient time to cause encapsulation of the agent into the liposomes. The external solution lacks a secondary or tertiary amine. The internal and external solutions are at substantially the same pH. In some embodiments, the secondary or tertiary amine is a secondary or tertiary alkylamine. The secondary or tertiary alkylamine can be an alkanolamine such as diethanolamine (DEA) or triethanolamine (TEA). In some embodiments, the internal solution further comprises a transition metal ion. In a particular embodiment, the transition metal ion is copper. The copper can be provided in a copper gluconate solution or a copper sulfate solution. The internal solution can further comprise a sodium gluconate solution or a gluconic acid solution. In some embodiments, the internal solution further comprises a phosphate or hydrochloric acid solution. The external aqueous solution comprises a pharmaceutically acceptable buffer. The external solution can comprise a phosphate or hydrochloric acid buffered solution. In a specific embodiment, the external solution is a sucrose/phosphate buffer at a neutral pH. The therapeutic agent can be a anthracycline, a campthothecin, or a vinca alkaloid. In some embodiments, the protonatable therapeutic agent is doxorubicin, daunorubicin, irinotecan, topotecan, vincristine or vinblastine. Sometimes, one or more second therapeutic agent(s) are added to the external solution simultaneously or sequentially relative to the therapeutic agent with the protonatable amino group. The second therapeutic agent can be one without a protonatable amino group. Typically, the liposomes are a mixture of 1,2-distearoyl-sn-glycero-3-phosphocholine (DSPC), 1,2-distearoyl-sn-glycero-3-phosphoglycerol sodium salt (DSPG), and cholesterol. In one embodiment, the mixture of DSPC, DSPG and cholesterol is in a molar ratio of 7:2:1.

Further provided herein is a liposomal composition prepared by the methods disclosed herewith.

In another aspect, provided herein is a liposomal composition comprising at least one therapeutic agent having a protonatable amino group; and a neutrally buffered secondary or tertiary amine. The secondary or tertiary amine can be a secondary or tertiary alkylamine. The neutrally buffered secondar or tertiary alkylamine can be an alkanolamine such as diethanolamine or triethanolamine. In particular embodiments, the therapeutic agent is irinotecan or daunorubicin. The composition can further comprising copper gluconate, sodium gluconate, or gluconic acid. Sometimes, the liposomes are a mixture 1,2-distearoyl-sn-glycero-3-phosphocholine (DSPC), 1,2-distearoyl-sn-glycero-3-phosphoglycerol sodium salt (DSPG), and cholesterol.

### BRIEF DESCRIPTION OF THE DRAWINGS

FIG. 1 shows the irinotecan to lipid ratio in liposomes containing 150 mM TEA/phosphate buffer, pH 7.0 inside and 300 mM sucrose/20 mM phosphate buffer, pH 7.0 outside. The loading of the drug was done at 50° C.

FIG. 2 shows the daunorubicin/lipid ratio in the liposomes containing () 220 mM TEA/HCl, pH 7.0 or (o) 220 mM TEA/100 mM sodium gluconate/HCl, pH 7.0 inside and 300 mM sucrose/20 mM phosphate/10 mM EDTA buffer outside. The loading of the drug was done at 50° C.

FIG. 3 shows the circular dichroism spectra of a solution of: (1) 2.5 mM irinotecan in water; (2) 2.5 mM copper gluconate/4.5 mM TEA; and (3) 2.5 mM irinotecan+2.5 mM copper gluconate/4.5 mM TEA. The solutions have a pH of 7.0. Spectra were recorded between 400 and 800 nm.

FIG. 4 shows the structure of irinotecan in its lactone form.

FIG. 5 shows the FTIR spectra of dry films of irinotecan from a solution in water. FIG. 5(A) shows the lactone form of irinotecan at pH 7.0; and FIG. 5(B) shows the carboxylate form of irinotecan at pH 8.7.

FIG. 6(A) shows the FTIR spectra of dry films from solutions in water of 11 mM irinotecan+11 mM copper gluconate/20 mM TEA (solid line), and the sum of the spectra of 11 mM irinotecan and 11 mM copper gluconate/20 mM TEA (dashed line). FIG. 6(B) shows the FTIR spectra of dry films from solutions in water of 11 mM irinotecan+11 mM copper gluconate/16 mM NaOH (solid line), and the sum of the spectra of 11 mM irinotecan and 11 mM copper gluconate/16 mM NaOH (dashed line).

FIG. 7 shows the absorption spectra of irinotecan in the presence of liposomes containing 100 mM copper gluconate/180 mM TEA (pH 7.0) inside and 300 mM sucrose/40 mM phosphate/10 mM EDTA buffer (pH 7.0) outside the liposomes. Samples were collected during the loading of the drug in the liposomes at 50° C. and quenched on ice. Aliquots were taken at the following timepoints: 0, 2, 5, 15, and 60 min. Spectra were recorded at room temperature.

FIG. 8 shows the emission spectra of irinotecan in the liposomes during its loading in the presence of liposomes containing 100 mM copper gluconate/180 mM TEA (pH 7.0) inside and 300 mM sucrose/40 mM phosphate/10 mM EDTA buffer (pH 7.0) outside at the following timepoints: 0, 2, 5, 15, and 60 min. The excitation wavelength was 400 nm. Emission spectra were collected between 425 and 650 nm. Each spectrum was recorded at room temperature.

FIG. 9 shows the emission spectra of irinotecan during its loading into the liposomes containing TEA phosphate buffer (150 mM TEA/95 mM phosphate, pH 7.0) inside and sucrose phosphate buffer (300 mM sucrose/20 mM phosphate, pH 7.0) outside, at the following timepoints: 0, 5, 30 and 60 min. Each spectrum was recorded at room temperature, at an excitation wavelength of 400 nm.

FIG. 10 shows the kinetic and stoichiometry correlation of TEA release (▪) with irinotecan uptake () for liposomes containing (A) 100 mM copper gluconate/180 mM TEA, pH 7.0 and (B) 10 mM sodium gluconate/180 mM TEA, pH 7.0.

FIG. 11 shows irinotecan/lipid molar ratios into liposomes containing 300 mM sucrose/40 mM phosphate/10 mM EDTA, pH 7.0 outside and the following internal buffers at pH 7.0: (♦) 100 mM copper gluconate/90 mM TEA; (▪) 100 mM copper gluconate/180 mM TEA and () 100 mM copper gluconate/270 mM TEA.

FIG. 12 shows the schematic of proposed neutral antiport exchange mechanism of irinotecan(ITN)/triethanolamine (TEA).

FIG. 13 shows irinotecan/lipid molar ratios in liposomes containing 100 mM copper gluconate/140 mM diethanolamine, pH 7.0 inside and 300 mM sucrose/20 mM phosphate/10 mM EDTA, pH 7.0 outside.

### MODES OF CARRYING OUT THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

Any suitable liposome may be useful in the methods and compositions provided herein. As used herein, the term “liposome” refers to vesicles comprised of one or more concentrically ordered lipid bilayers encapsulating an aqueous phase. Typically, liposomes are formed from standard vesicle-forming lipids, which generally include neutral and negatively charged phospholipids and a sterol, such as cholesterol. The selection of lipids is generally guided by consideration of, e.g., liposome size and stability of the liposomes in the bloodstream. Liposomes can be unilamellar or multilamellar vesicles.

The liposomes can be prepared by any suitable technique. See, e.g., Torchillin et al. (eds), LIPOSOMES: A PRACTICAL APPROACH (Oxford University Press 2nd Ed. 2003). Exemplary techniques include but not limited to lipid film/hydration, reverse phase evaporation, detergent dialysis, freeze/thaw, homogenation, solvent dilution and extrusion procedures. In some embodiments, the liposomes are generated by extrusion procedures as described by Hope, et al., Biochim. Biophys. Acta (1984) 55-64 or as set forth in the Examples below.

In one embodiment, the liposomes are a mixture of 1,2-distearoyl-sn-glycero-3-phosphocholine (DSPC), 1,2-distearoyl-sn-glycero-3-phosphoglycerol sodium salt (DSPG), and cholesterol. In a specific embodiment, the mixture of DSPC, DSPG and cholesterol is in a molar ratio of 7:2:1.

The method provided herein employ liposomes with an internal (intraliposomal) aqueous solution or medium that comprises a neutrally buffered secondary or tertiary amine solution. Any suitable secondary or tertiary amines can be employed, particularly those useful in pharmaceutical formulations. For example, a secondary or tertiary alkylamine can be used. Suitable alkylamines include substituted amine such as secondary or tertiary alkanolamines. In one embodiment, the alkanolamine is triethanolamine (TEA) or diethanolamine (DEA). Any suitable molar concentration of the secondary or tertiary amine can be employed. Exemplary molar concentrations can vary from about 5 mM to 500 mM, sometimes 50 mM to 300 mM, often 100-300 mM. Any suitable means of buffering can be employed that maintains the solution at a neutral pH, preferably pH 7. Typically, phosphate (e.g., phosphoric acid) or hydrochloric acid are used. The internal aqueous solution can also comprise additional components such as sodium gluconate and gluconic acid.

In some embodiments, the internal aqueous solution includes a transition metal ion. Any suitable transition metal ion can be employed. In one embodiment, the transition ion is copper. In some embodiments, the internal aqueous solution can further comprise a copper gluconate solution or a copper sulfate solution. Any suitable ratio of transition metal ion to drug may be employed. For example, the ratio may range from 5:1 to 1:5 transition metal ion:drug.

The external (extraliposomal) aqueous solution or buffer is a pharmaceutically acceptable buffer at substantially the same pH as the internal aqueous solution. The external solution initially lacks any secondary or teritiary amines when first added to the liposome mixture. The external solution can comprise any suitable buffering agent that keeps the solution at a neutral pH, preferably pH 7. Such buffering agents include but are not limited to phosphate or hydrochloric acid. In some embodiments, the external aqueous solution can also contains additional buffer components that are cryoprotective, increase stability, and the like. For example, the external aqueous solution can include sucrose.

The pH of the internal and external aqueous solutions are substantially the same and are neutral, i.e., about pH 7. Thus, the pH can range from 6.5 to 7.4. In some embodiments, the pH of the internal and external aqueous solutions are pH 7.0.

For loading or encapsulating the drug, the liposomes having an internal aqueous solution with a neutrally buffered secondary or tertiary amine aqueous solution are placed in an external aqueous solution, where each of the solutions a neutral pH that is substantially the same. The drug is added in the external solution lacking a secondary or tertiary amine on the outside of the liposome. At a neutral pH, the drug with the protonatable amino group diffuses through the phospholipid bilayer in its neutral form while the neutral form of secondary or tertiary amine permeates towards the extraliposomal medium in a manner that is kinetically and stoichiometrically correlated to drug uptake. Upon movement of the uncharged form of secondary or tertiary amine from inside the liposome, the equilibrium of secondary or tertiary amine will shift to reprotonate secondary or tertiary amine in the extraliposomal medium and deprotonate secondary or tertiary amine in the liposome interior, resulting in a transbilayer movement of uncharged molecules followed by protonation and deprotonation. This creates a mutually self-buffered system where both secondary or tertiary amine and drug can readily convert between protonated and deprotonated forms to similar extents, thereby allowing active transbilayer transport without generating unfavorable electrochemical gradients that would impede further transmembrane flux of either secondary or tertiary amine or the drug.

The therapeutic agent useful in the disclosed liposomes and associated methods has a protonatable amino group. A therapeutic agent is one that is biologically active. Such agent are typically small molecule drugs useful in the treatment of neoplasms or infectious diseases. Exemplary drugs include anthracyclines, campthothecins, and vinca alkaloids. Specific drugs suitable in the disclosed liposomes are doxorubicin, daunorubicin, irinotecan, topotecan, vincristine and vinblastine. Other exemplary therapeutic agents include those disclosed in U.S. Pat. No. 5,785,987.

In addition to loading a single therapeutic agent, the method can be used to load multiple therapeutic agents, either simultaneously or sequentially, by placing one or more additional therapeutic agents in the external aqueous solution. The additional therapeutic agent is one whose activity complements the desired activity of the therapeutic agent with the protonatable amino group. The additional therapeutic agent may have a protonatable amino group but is not required to have one. Typically, the second therapeutic agent does not have a protonatable amino group. Thus, the mode of encapsulation for the additional therapeutic agent may differ from the mode of encapsulation for the therapeutic agent with the protonatable amino group. Additional agents can include but are not limited to a pharmaceutical agent, such as a chemotherapeutic drug or a toxin; a bioagent such as a cytokine or ligand; or a radioactive moiety.

The present invention also provides liposomes and therapeutic agents in kit form. The kit will typically be comprised of a container which is compartmentalized for holding the various elements of the kit. The therapeutic agents which are used in the kit are those agents which have been described above. In one embodiment, one compartment will contain a second kit for loading a therapeutic agent into a liposome just prior to use. Thus, the first compartment will contain a suitable agent in a neutral buffer which is used to provide an external medium for the liposomes, typically in dehydrated form in a first compartment. In other embodiments, the kit will contain the compositions of the present inventions, preferably in dehydrated form, with instructions for their rehydration and administration. In still other embodiments, the liposomes and/or compositions comprising liposomes will have a targeting moiety attached to the surface of the liposome.

The liposomes of the present invention may be administered to warm-blooded animals, including humans. These liposome and lipid carrier compositions may be used to treat a variety of diseases in warm-blooded animals. Examples of medical uses of the compositions of the present invention include but are not limited to treating cancer, treating cardiovascular diseases such as hypertension, cardiac arrhythmia and restenosis, treating bacterial, fungal or parasitic infections, treating and/or preventing diseases through the use of the compositions of the present inventions as vaccines, treating inflammation or treating autoimmune diseases. For treatment of human ailments, a qualified physician will determine how the compositions of the present invention should be utilized with respect to dose, schedule and route of administration using established protocols. Such applications may also utilize dose escalation should bioactive agents encapsulated in liposomes and lipid carriers of the present invention exhibit reduced toxicity to healthy tissues of the subject.

Pharmaceutical compositions comprising the liposomes of the invention are prepared according to standard techniques and further comprise a pharmaceutically acceptable carrier. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the liposome suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of liposomes, in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration may be increased to lower the fluid load associated with treatment. Alternatively, liposomes composed of irritating lipids may be diluted to low concentrations to lessen inflammation at the site of administration. For diagnosis, the amount of liposomes administered will depend upon the particular label used, the disease state being diagnosed and the judgment of the clinician but will generally be between about 0.01 and about 50 mg per kilogram of body weight, preferably between about 0.1 and about 5 mg/kg of body weight.

Preferably, the pharmaceutical compositions are administered intravenously. Typically, the formulations will comprise a solution of the liposomes suspended in an acceptable carrier, preferably an aqueous carrier. A variety of aqueous carriers may be used, e.g., water, buffered water, 0.9% isotonic saline, 5% dextrose and the like. These compositions may be sterilized by conventional, well known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, EDTA, etc.

Dosage for the liposome formulations will depend on the ratio of drug to lipid and the administrating physician's opinion based on age, weight, and condition of the patient.

The methods of the present invention may be practiced in a variety of hosts. Preferred hosts include mammalian species, such as humans, non-human primates, dogs, cats, cattle, horses, sheep, and the like.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLE 1

### Liposomal Encapsulation of Irinotecan and Daunorubicin Under Neutral Conditions

The encapsulation efficiency for therapeutic agents with protonatable amino groups was investigated using a neutrally buffered system in the presence of a tertiary amine. Liposomes were prepared with a neutral internal aqueous solution comprising triethanolamine. Irinotecan or daunorubicin were prepared in a sucrose phosphate buffer at pH 7.0. The efficiency of drug encapsulation by liposomes with a neutral internal and external solution were then examined.

The liposomes were prepared using phospholipids and cholesterol dissolved in chloroform/methanol/water (95/4/1) at a molar ratio of 7:2:1 for DSPC:DSPG:Chol. The lipids were labeled with trace amounts of ³H-cholesteryl hexadecyl ether, a non-exchangeable, non-metabolizeable lipid marker to allow liposome quantitation by scintillation counting. The solvent was evaporated under a stream of nitrogen and dried under vacuum for at least 4 hours. The sample was then hydrated with either 100 mM copper gluconate or sucrose phosphate buffer (300 mM sucrose, 20 mM phosphate, pH 7.0) to obtain a final lipid concentration of 50 mg/ml. The liposomes were then extruded ten times at 70° C. through two polycarbonate filters with pores diameters of 0.1 μm at moderate pressure using a liposome extruder (Lipex Inc., Vancouver, BC). For copper containing liposomes, the external copper gluconate was exchanged with a 300 mM sucrose/20 mM phosphate/10 mM EDTA (pH 7.0) by tangential flow dialysis. The mean size distribution of the resulting large unilamellar vesicles (80-120 nm) was determined using a Nicomp submicron particle sizer model 370 (Nicomp, Santa Barbara, Calif.).

For irinotecan loading, the solutions of irinotecan were made by dissolving the drug either in water at 50° C. or in sucrose phosphate buffer (300 mM sucrose, 40 mM phosphate) at room temperature. When necessary, the pH of the solution was adjusted to the desired value using NaOH. The final concentration of irinotecan was 15 mM. The 100 mM copper gluconate buffer was prepared by dissolving the copper gluconate powder in water at room temperature and adjusting the pH to 7.0 using NaOH or TEA. The final concentration of TEA required to buffer the solution of copper gluconate to pH 7.0 was 180 mM. For solutions of copper gluconate with 90 mM and 270 mM TEA, the pH was brought to 7.0 with NaOH and HCl, respectively. The solution of 10 mM sodium gluconate/180 mM TEA was made by dissolving the sodium gluconate in water, adding TEA and finally adjusting the pH to 7.0 with HCl. Mixtures of irinotecan and copper gluconate/TEA were made to obtain a drug:metal molar ratio of 1:1. Further addition of irinotecan to copper gluconate/TEA at higher drug:metal ratios caused the formation of a precipitate in the solution. The precipitate was isolated by centrifugation (12000 rpm, 15 min) and was solubilized with 2 mM EDTA in water.

Similar preparations were employed for daunorubicin.

For liposomal loading, the drug solution and the liposomes were incubated separately at 50° C. for approximately five minutes to equilibrate the temperature. The two solutions were combined to obtain a 0.2:1 drug to lipid molar ratio; aliquots were removed at various time points and put on ice. Aliquots of 75 μl were applied to a Sephadex G-50 spin column. The columns were prepared by adding glass wool to a 1 ml syringe and Sephadex G-50 beads hydrated in sucrose phosphate buffer (300 mM sucrose, 40 mM phosphate, pH 7.0). The columns were packed by spinning at 290×g for 1 minute. Following addition of the sample to the column, the liposome fraction was collected in the void volume by centrifuging at 515×g for 1 minute. Aliquots of the spin column eluant and the pre-column solution were taken and analyzed by liquid scintillation counting to determine the lipid concentration at each time point. The irinotecan concentration in each liposomal fraction was determined using a UV-based assay. Briefly, a 100 μl aliquot of each liposomal sample (or smaller volume adjusted to 100 μl with distilled water) was solubilized in 100 μl of 10% Triton X-100 plus 800 μl of 50 mM citrate/trisodium citrate, 15 mM EDTA, pH 5.5 and heated in boiling water until the cloud point was reached. The samples were cooled to ambient temperature. The absorbance at 370 nm was measured and compared to a standard curve. The concentration of TEA was determined by HPLC.

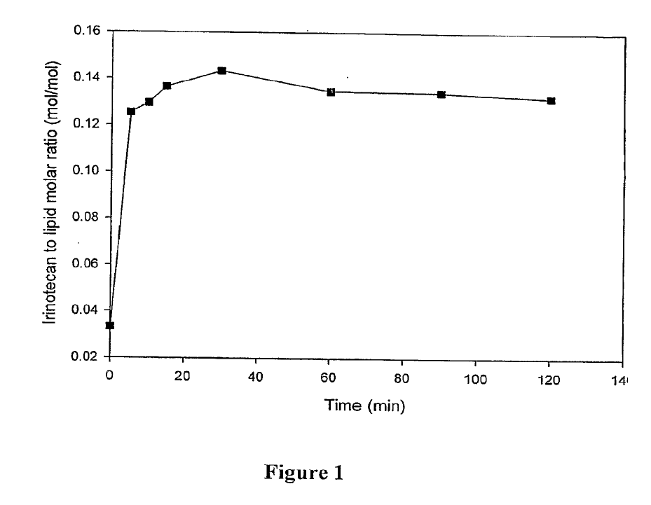

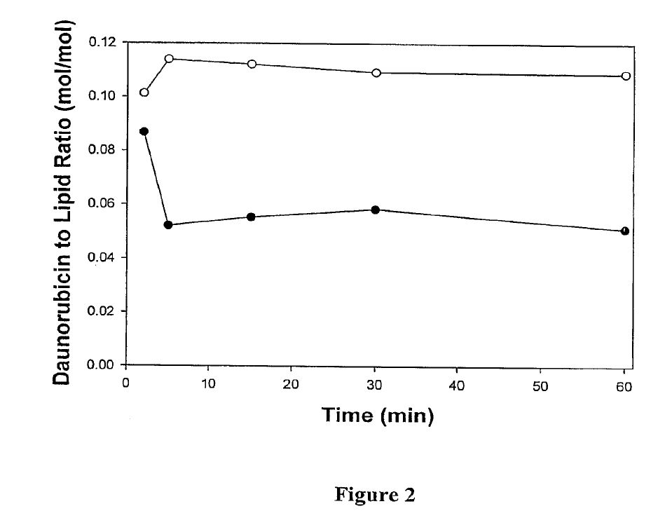

Using a TEA buffered internal solution at pH 7.0 and an external phosphate buffer, pH 7.0, liposomes were successfully loaded with irinotecan. (FIG. 1). Likewise, liposomes successfully encapsulated daunorubicin using either a triethanolamine hydrochloride internal solution, pH 7.0 or a triethanolamine/sodium gluconate/HCl solution, pH 7.0. (FIG. 2).

### EXAMPLE 2

### Copper Gluconate/Triethanolamine Interact with Irinotecan

To investigate the role of copper, triethanolamine and irinotecan during liposomal loading, their molecular interactions were analyzed using CD dichroism, FTIR analysis, UV/VIS and fluorescence spectroscopy. The approach was to first characterize the interaction between irinotecan and copper gluconate/TEA in solution at a 1:1 molar ratio using CD and FTIR spectroscopy. The interaction between irinotecan and copper gluconate/TEA in the liposomes was then characterized as this allowed the examination of the interaction between the drug and the metal at high intra-liposomal concentrations that reflected conditions used for the loading of irinotecan.

Circular dichroism analyses were conducted using a Jasco J-810 spectropolarimeter, calibrated with a solution of 1% d-camphor-10-sulfonic acid in water. All spectra were recorded at 25° C. between 190 and 800 nm using a quartz cell with a I cm or a 0.2 cm path length. For each spectrum, 2 scans were accumulated at a scanning speed of 50 nm/min.

FTIR measurements were made at room temperature in transmission mode using a Nicolet Nexus 870 spectrometer (Nicolet Instrument, Madison, Wis., USA) equipped with a liquid nitrogen-cooled mercury cadmium telluride detector. Spectra of dry films of irinotecan and irinotecan/copper mixtures were obtained by spreading 20 μl of the sample on a BaF₂ window (Wilmad Glass Co. Inc. Buena, N.J.). The sample was dried with a stream of nitrogen and left overnight in a desiccator before recording the spectra. For each spectrum, 250 scans were co-added at a 4 cm⁻¹ resolution, using a Happ-Genzel apodization. Data analysis was done using the Grams AI software (Galactic Industries, Salem, N.H., USA). The second derivative of the spectra was performed to determine the frequency of the components of unresolved bands.

For UV/VIS and fluorescence spectroscopy, samples were prepared using the aliquots taken during the loading process, as described above, before applying to the Sephadex G-50 spin columns. The aliquots were diluted in sucrose phosphate buffer (300 mM sucrose, 40 mM phosphate, pH 7.0) to obtain a final irinotecan concentration of 6 μM. The same solutions were used for both UV-Vis and fluorescence measurements. The spectra of the liposomes alone were not subtracted from the spectra of the mixtures because the contribution of the liposomal signal to that of the drug was found to be negligible. UV-Vis spectra were recorded with a Shimadzu 2401-PC spectrophotometer (Shimadzu Scientific Instruments, Columbia, Md.). Fluorescence spectra were recorded using either a PerkinElmer (model LS 50B, PerkinElmer Life and Analytical Sciences, Woodbridge, ON) or a Varian Cary Eclipse (Varian, Palo Alto, Calif.) spectrofluorometers. For fluorescence measurements, the excitation wavelength was set at 400 nm and the emission scans were obtained from 425 to 650 nm. The slits were set at 2.5 nm. Measurements were made at ambient temperature using a quartz cell with a 1 cm path length.

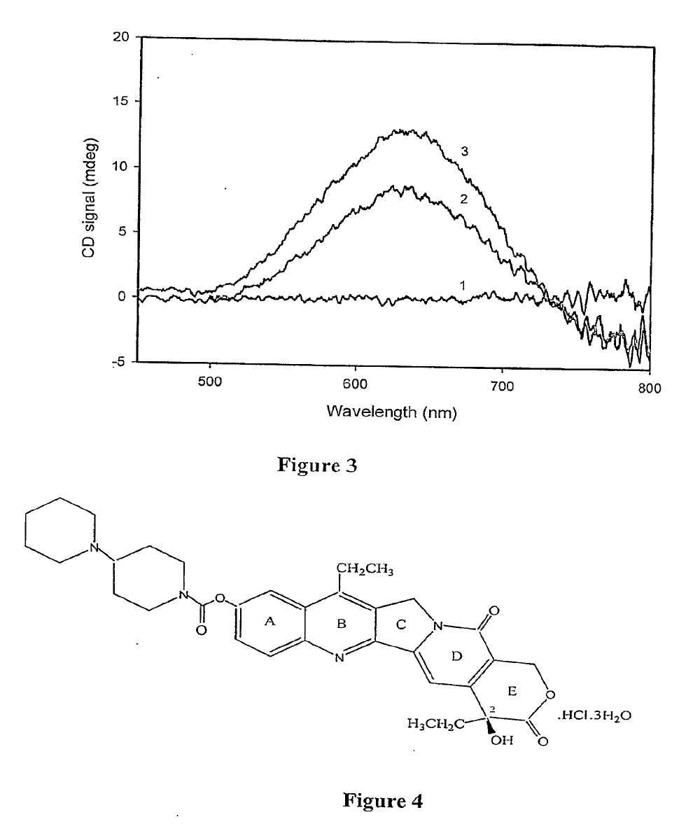

The CD spectrum of a 2.5 mM solution of copper gluconate buffered to 7.0 with 4.5 mM TEA exhibited a broad band centered at 630 nm whose intensity increased from 8 to 13 mdeg upon addition of 1 mole-equivalent of irinotecan to copper gluconate/TEA (FIG. 3). Since irinotecan does not have a CD signal in the visible wavelength range, the increase in intensity of the CD signal of copper gluconate suggested an interaction between the drug and copper gluconate/TEA.

Since irinotecan has a chiral center located on carbon 2 of the lactone ring (FIG. 4), the possibility of characterizing the interaction by looking at changes in the CD signal of irinotecan was investigated. At drug concentrations greater than 250 μM, the high absorption of irinotecan induced artifacts in its CD signal. Therefore, spectra were recorded using low concentrations of the drug. The CD spectrum of irinotecan at 250 μM exhibited two conservative CD signals in the UV region. Addition of copper gluconate/TEA (pH 7.0) to irinotecan at a 1:1 molar ratio did not induce any change to the CD spectrum of the drug. However, it is possible that the low drug concentration precluded monitoring the interaction of copper gluconate/TEA with irinotecan in contrast to the high irinotecan concentrations inside the liposomes upon encapsulation (>50 mM). This is supported by the fact that at neutral pH, concentrated solutions of irinotecan:copper gluconate/TEA at molar ratios higher than 1:1 caused the formation of a blue precipitate. Analysis of the precipitate by atomic absorption and HPLC revealed that the stoichiometry of irinotecan:copper in the precipitate was 1:5. The formation of a precipitate provides further evidence of an interaction between copper gluconate/TEA and irinotecan.

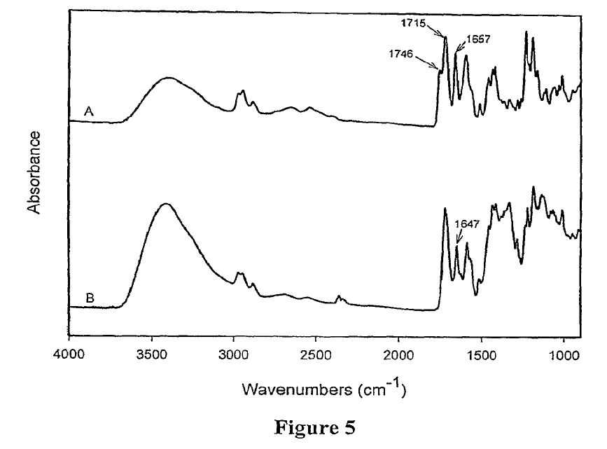

Vibrational spectroscopy was used to further investigate the potential interaction between irinotecan and copper in free solution. FIG. 5 shows the spectra of irinotecan at pH 7.0 and pH 8.7. Since irinotecan has several possible binding sites, tentative assignment of the bands of the spectra to its functional groups was performed in order to identify which group is involved in an interaction with copper gluconate/TEA. The C═O stretching absorption bands appear in the region of 1870-1540 cm⁻¹. The position of the carbonyl bands is affected by several factors including intermolecular and intramolecular hydrogen bonding. The band at 1746 cm⁻¹ is attributable to the C═O stretching vibration of the carbonyl group of the lactone ring (FIG. 4, ring E) since it is absent in the spectrum of irinotecan at pH 8.7 where the drug exists primarily in the carboxylate form. This conversion to the carboxylate form was confirmed by HPLC analysis.

Under experimental conditions at pH 7.0, irinotecan was found to be predominantly in its lactone form (data not shown). The band at 1715 cm⁻¹ is assignable to the carbonyl group attached to quinoline moiety (FIGS. 4 and 5) and was not affected by the hydrolysis of the lactone. When the drug is in its carboxylate form, the carbonyl group of ring D (see FIGS. 4 and 5) is involved in hydrogen bonding interactions with the neighboring hydroxyl group, formed upon opening of the ring. This hydrogen bond caused a shift of the band at 1657 cm⁻¹ to lower frequencies, which appears at 1647 cm⁻¹ on the spectrum of irinotecan at pH 8.7. Thus, the band at 1657 cm⁻¹ on the spectrum of irinotecan at pH 7.0 was assigned to the carbonyl group of the pyridone moiety (FIG. 4, ring D). At neutral pH, addition of copper gluconate/TEA to irinotecan at a 1:1 molar ratio does not affect the three carbonyl groups of the drug. This indicates that the interaction between irinotecan and copper gluconate/TEA likely occurs through other groups on the molecule.

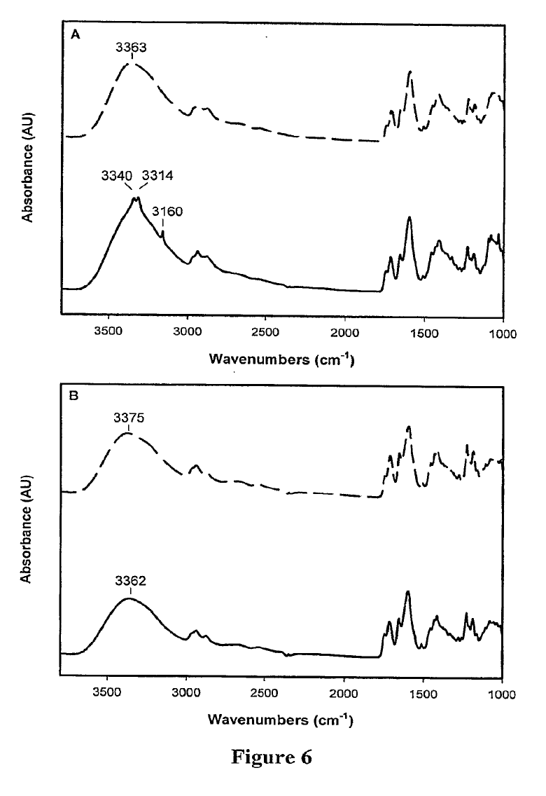

The resulting spectrum obtained from the sum of the spectra of copper gluconate/TEA and irinotecan was compared to that of the mixture of irinotecan and copper gluconate/TEA at the same relative concentrations. A lack of interaction between the two compounds would result in similar spectra with bands appearing at the same frequency. FIG. 6A shows that when 11 mM copper gluconate/20 mM TEA is added to 11 mM irinotecan, the band due to the hydroxyl stretching vibration at 3363 cm⁻¹ is split and shifted to lower frequencies (3340-3314 cm⁻¹). The two components indicate the presence of two populations of hydroxyl groups. Comparison of this spectrum to that of irinotecan/TEA revealed that the band at 3314 cm⁻¹ and the sharp peak at 3160 cm⁻¹ are due to TEA. The band at 3340 cm⁻¹ is attributable to irinotecan hydrogen bonded with TEA. FIG. 6B compares the spectrum of irinotecan/copper gluconate/NaOH (11/11/16 mM, respectively) to that of the sum of the spectra of irinotecan and copper gluconate/NaOH. Contrary to what was observed above for irinotecan/copper gluconate/TEA, no splitting of the hydroxyl band occurred, suggesting a homogenous population of hydroxyl groups. This is consistent with the absence of TEA in that sample. The hydroxyl band appeared at a slightly lower frequency in the mixture (3362 cm⁻¹) than in the single spectra (3375 cm⁻¹). This indicates a strengthening of the hydrogen bonds with the hydroxyl groups.

The above results indicate that in solution, irinotecan is capable of interacting with copper gluconate/TEA. However, the concentrations of irinotecan and copper gluconate/TEA possible in solution do not approximate the conditions of the formulation where the intra-liposomal drug concentrations can exceed 50 mM. Also, the nature of the interactions could be modulated by the presence of the lipid bilayer. Therefore, UV/VIS and fluorescence spectroscopy were used to investigate the interaction between irinotecan and copper gluconate under conditions where irinotecan was encapsulated inside liposomes containing 100 mM copper gluconate/180 mM TEA, pH 7.0. It should be noted that analysis of irinotecan/copper gluconate/TEA containing liposomes by cryo-electron microscopy did not reveal any morphological features that were distinct from liposomes containing only copper gluconate/TEA. There was no evidence of irinotecan crystallization or precipitation inside the drug loaded liposomes and also no apparent changes in the membrane structure when the liposomes are loaded with drug. In both cases, the liposomes exhibited a faceted morphology with corners, edges and textured membrane surfaces, consistent with gel phase liposomes containing low amounts of cholesterol.

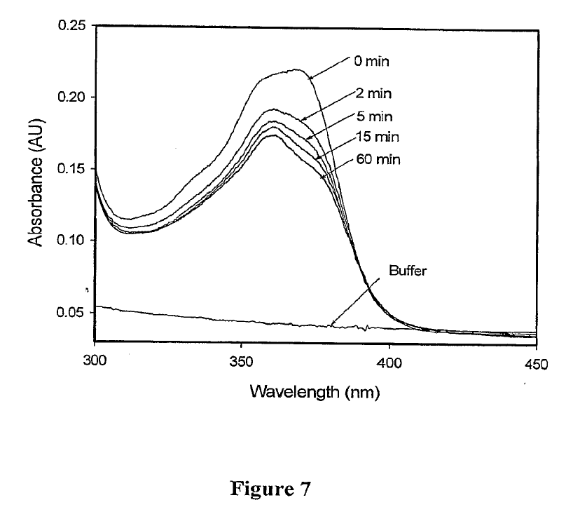

The absorption spectra of irinotecan in the presence of liposomes containing copper gluconate/TEA, pH 7.0 is shown in FIG. 7. The spectra were recorded from samples collected at different timepoints during the loading of irinotecan into the liposomes at 50° C. They are similar to the spectra of irinotecan in free solution and are characterized by four bands appearing at approximately 220, 255, 358 and 370 nm. Only the region between 280 nm and 440 nm is shown in FIG. 7 since changes in the spectra below this region were negligible. The absorbance spectra were not corrected for background scattering due to the low absorbance of drug-free liposomes in this wavelength range. When the drug was incubated with liposomes containing copper gluconate/TEA, drug encapsulation occurred. The UV-VIS spectra showed that the bands at 358 and 370 nm shifted to 360 and 378 nm, respectively, and were accompanied by a decrease in intensity of the absorption band at 370 nm of irinotecan by approximately 25% (FIG. 7).

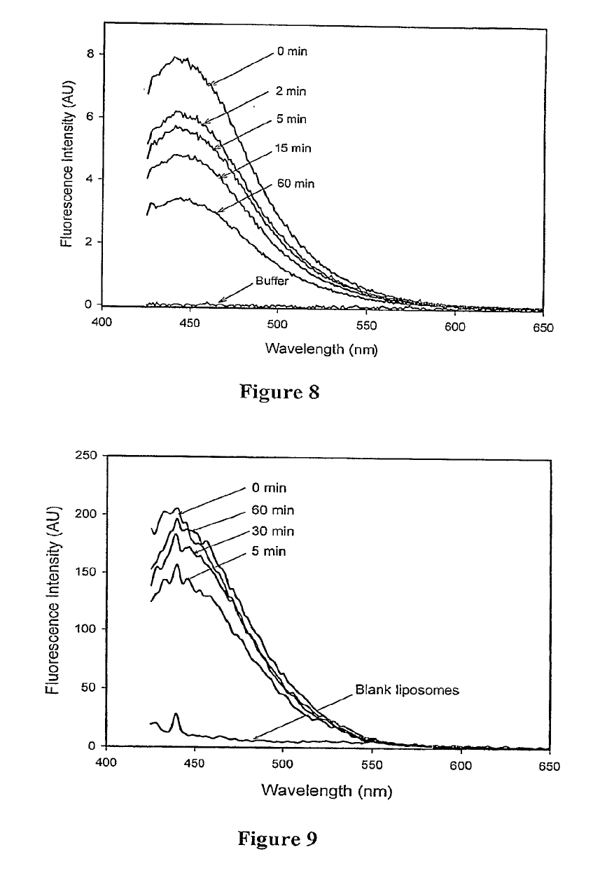

The fluorescence of irinotecan was also monitored at various time points during the irinotecan loading process. When irinotecan was added to liposomes containing 100 mM copper gluconate/180 mM TEA, pH 7.0, a 60% decrease of the fluorescence intensity at 440 nm occurred within 1 h without any apparent shift of the peak wavelength (FIG. 8). It should be noted that the fluorescence intensity of irinotecan increased by approximately 15% over 60 min when incubated with liposomes containing sucrose phosphate buffer that were not able to accumulate irinotecan. In addition, the emission intensity of irinotecan in a solution of sucrose phosphate buffer at 50° C. decreased by approximately 8% in the first 5 min and then stabilized.

The data indicate that drug loading was negligible when NaOH was used to raise the pH of copper gluconate to 7.0. Thus, irinotecan fluorescence was monitored in the presence of liposomes containing copper gluconate/NaOH following the loading method described above. The results indicate that in the presence of 100 mM copper gluconate/149 mM NaOH, the fluorescence intensity of irinotecan increased by 20% over 60 minutes at 50° C. These small changes are similar to those observed above for copper-free liposomes incubated with irinotecan and in contrast to the quenching that occurred in the liposomes containing copper gluconate/TEA. These results suggest that the presence of TEA is necessary to induce the loading of irinotecan.

The fluorescence intensity of irinotecan was monitored in the presence of liposomes containing TEA/phosphate buffer (150 mM TEA, 95 mM phosphate, pH 7.0). The emission intensity of irinotecan added to the liposomes at a 0.2:1 drug to lipid ratio (mol:mol) decreased by 25% within 5 minutes then gradually increased to near the original fluorescence intensity within 60 min at 50° C. (FIG. 9). Interestingly, drug encapsulation occurred and stabilized at approximately 70% efficiency, similar to that was observed above with copper gluconate/TEA containing liposomes (FIG. 1). Room temperature dialysis of the TEA/phosphate encapsulated irinotecan resulted in drug release whereas copper gluconate/TEA liposomes exhibited no drug release over 24 hr.

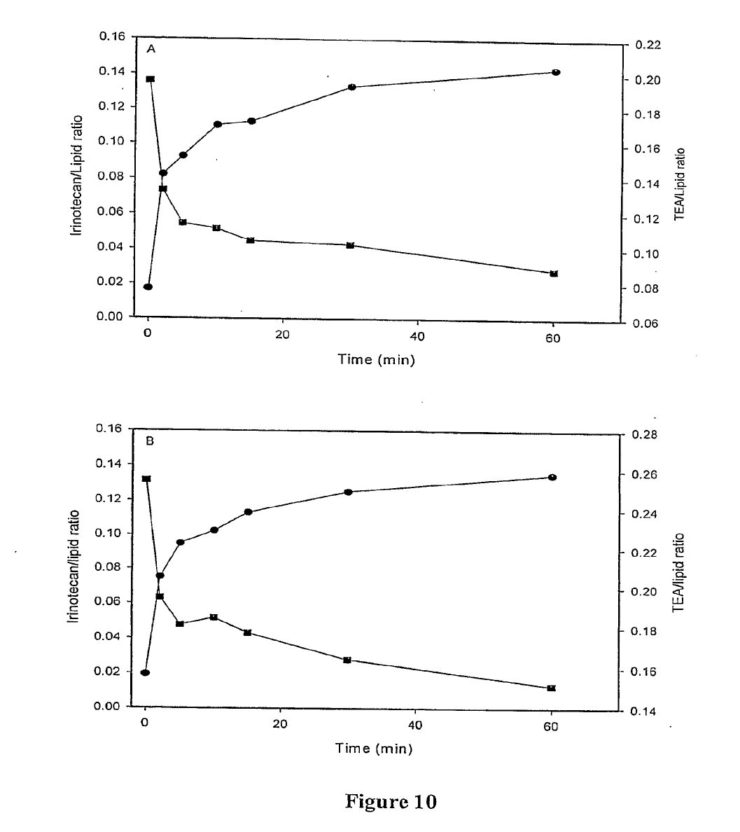

The role of copper in inducing drug fluorescence quenching was assessed by adding irinotecan to liposomes containing 10 mM sodium gluconate/180 mM TEA, pH 7.0. Contrary to what was observed above for liposomes containing TEA/phosphate or sucrose phosphate buffer, drug fluorescence quenching occurred. Similarly to copper gluconate/TEA containing liposomes, irinotecan encapsulation occurred and stabilized at 70% efficiency (FIG. 10B).

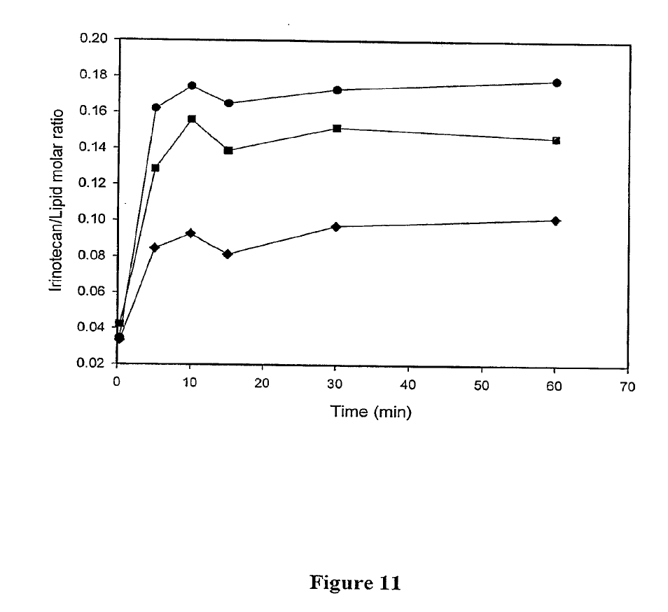

To further investigate the role of TEA in irinotecan loading, the liposome encapsulated TEA concentration relative to that of irinotecan was monitored during the encapsulation process over 1 h at 50° C. TEA/lipid ratios decreased (reflecting release from the liposomes) by 0.08 μmol TEA/μmol lipid after 2 min and approximately 0.11 μmol TEA/μmol lipid after 1 h. In comparison, irinotecan/lipid molar ratios increased by 0.08 μmol irinotecan/μmol lipid and 0.13 μmol irinotecan/μmol lipid after 2 and 60 min, respectively (FIG. 10). This observation established a kinetic and stoichiometric relationship between irinotecan encapsulation and TEA efflux. This was further supported that the fact that the amount of irinotecan encapsulated could be controlled by the amount of TEA inside the liposomes. FIG. 11 demonstrates that decreasing the concentration of TEA to 90 mM reduced the amount of drug loading by 50% while approximately 90% irinotecan encapsulation was obtained when the concentration of TEA was increased to 270 mM.

In free solution, the data indicated that at pH 7.0 the CD signal of copper gluconate/TEA increased upon addition of irinotecan. The CD signal of copper gluconate has been proposed to result from the contribution of one C(S)—OH and two C(R)—OH groups. Since the binding of a chiral molecule to copper is expected to enhance the CD signal, the increase in intensity of the CD band may result from the contribution of irinotecan to the chirality of copper gluconate/TEA. This could occur either by the binding of irinotecan to the copper center or to one of its ligands such as gluconate and/or TEA. FTIR data showed that irinotecan was involved in hydrogen bonding interactions with TEA. Taken together, the above observations did not reveal any evidence of irinotecan binding to copper but indicated that irinotecan interacted with TEA.

When liposomes containing copper gluconate/TEA were incubated with irinotecan under conditions that promote drug encapsulation, a quenching of irinotecan fluorescence was observed. For liposomes containing sucrose phosphate buffer, no drug encapsulation was obtained and a slight increase in the fluorescence emission intensity of irinotecan was seen. This latter change is consistent with a passive relocation of a portion of the drug in a more hydrophobic environment with a lower dielectric constant and is likely the result of irinotecan partitioning into the membrane.

When copper gluconate was pH adjusted with NaOH and trapped inside liposomes, no loading of irinotecan was observed and no quenching in irinotecan fluorescence occurred. On the contrary, the emission intensity increased. Since neither loading nor quenching of the fluorescence were observed with copper gluconate/NaOH solutions, the presence of TEA appeared to be required for the loading of irinotecan. This is supported by the observation that accumulation of irinotecan inside the liposomes was shown to be kinetically as well as stoichiometrically correlated with TEA efflux (FIG. 10 A and B).

While not being bound by theory, one scenario that could account for the encapsulation of irinotecan inside liposomes containing copper gluconate/TEA. Gluconate is tightly bound to copper (Kₐ=1.95×10¹⁸) through its carboxyl and hydroxyl moieties as previously reported [26, 35]. Upon buffering of the solution with TEA, the nitrogen and/or hydroxyl groups of TEA could bind to copper. When irinotecan is added to the outside of the liposome the drug diffuses through the phospholipid bilayer in the neutral lactone form while the neutral form of TEA permeates towards the extraliposomal medium in a manner that is kinetically and stoichiometrically correlated to irinotecan uptake. At pH 7.0, based on a pKa of 7.8 for TEA, the ratio of uncharged to charged molecules is 1:6.3. Upon movement of the uncharged form of TEA from inside the liposome, the equilibrium of TEA will shift to reprotonate TEA in the extraliposomal medium and deprotonate TEA in the liposome interior.

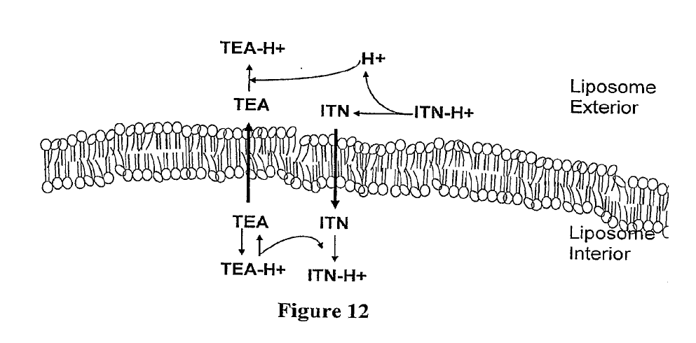

Likewise, as irinotecan has a pKa of 8.1, it also has a significant population of both charged and uncharged molecules at pH 7.0. The ratio of uncharged to charged molecules of irinotecan at pH 7.0 is 1:12.6 and the same phenomenon of transbilayer movement of uncharged molecules followed by protonation and deprotonation may be expected to occur, but in the opposite orientation relative to TEA. This creates a mutually self-buffered system where both TEA and irinotecan can readily convert between protonated and deprotonated forms to similar extents, thereby allowing active transbilayer transport without generating unfavorable electrochemical gradients that would impede further transmembrane flux of either TEA or irinotecan. A schematic representation of this proposed irinotecan/TEA neutral antiport exchange is shown in FIG. 12.

Regardless of the liposomal location of the drug complex, it appears that irinotecan interacts with neighboring drug molecules resulting in larger supramolecular complexes which could result in the fluorescence quenching of irinotecan after encapsulation. Such copper gluconate/TEA induced aggregates of the drug could stabilize irinotecan in its lactone form which would account for the high lactone content inside the copper gluconate/TEA containing liposomes at pH 7.0 where significant carboxylate content would otherwise be expected. Copper gluconate may play a role in modulating the flux of irinotecan and TEA across the liposomal bilayer and also appears to be important in controlling the release of irinotecan in vivo.

### EXAMPLE 3

### Encapsulation of Irinotecan Using Diethanolamine Buffered Liposomes

The encapsulation efficiency using a neutrally buffered system comprising a secondary amine in the presence of a therapeutic agent with protonatable amino group also was examined. Liposomes were prepared with a neutral internal aqueous solution comprising diethanolamine. The efficiency of irinotecan encapsulation by liposomes with a neutral internal solution buffered by diethanolamine and a neutral external aqueous solution was then examined.

DSPC, cholesterol and DSPG were weighed out into capped scintillation vials. DSPC was dissolved in chloroform at 60 mg/ml, cholesterol was dissolved in chloroform at 25 mg/ml, and DSPG was dissolved in chloroform:methanol:water (50/10/1) at 30 mg/ml. The lipids were then combined in the appropriate proportions. The lipid mixtures were each radiolabeled with 1 μCi ³H-CHE while still in solvent. A stream of N₂ gas, while heating the mixture, was used to remove solvent. The resulting lipid films were left under vacuum for a few minutes, then redissolved in chloroform. The drying process was then repeated, and the lipid films were allowed to dry on a vacuum pump for 4+hours. The lipid film was rehydrated in 2 mL 100 mM copper gluconate, 140 mM diethanolamine, pH 7.0 and aliquots of known volume were taken (just before extrusion, when lipids are MLVs) to determine the specific activity of each lipid mixture. MLVs were extruded at 70° C. through two 100 nm filters for a total of eight passes without difficulty. The liposomes were then allowed to cool down to room temperature.

Samples were buffer exchanged into 300 mM sucrose/20 mM phosphate/10 mM EDTA, pH 7.0 by tangential flow.

Irinotecan loading at 50° C. was attempted with a target molar Irinotecan to lipid ratio of 0.1. Clinical material of Irinotecan was used for a Irinotecan stock. Drug and liposome samples were pre-heated separately at 50° C. for 5 minutes, then combined at t=0. After 2, 5, 10, 15, 30, and 60 minutes of incubation, spun column samples were taken by placing 100 μl Hepes buffered saline, pH 7.4 onto a spin column, then 100 μL sample. The spin columns were then centrifuged for 1 minute at 1800 rpm (652 ref). Samples of the spun column eluant and the pre-column solution were counted by liquid scintillation counting to determine the lipid concentration at each time point. Irinotecan concentrations were determined using a UV assay. Briefly, 100 μL sample+100 μL 10% Triton X-100+800 μL 10 mM citric acid, 50 mM sodium citrate, 15 mM EDTA, pH 5.5. Samples are heated to cloud point using boiling water, then cooled to room temperature using tap water. Irinotecan is quantitated by absorbance at 370 nm against a standard curve.

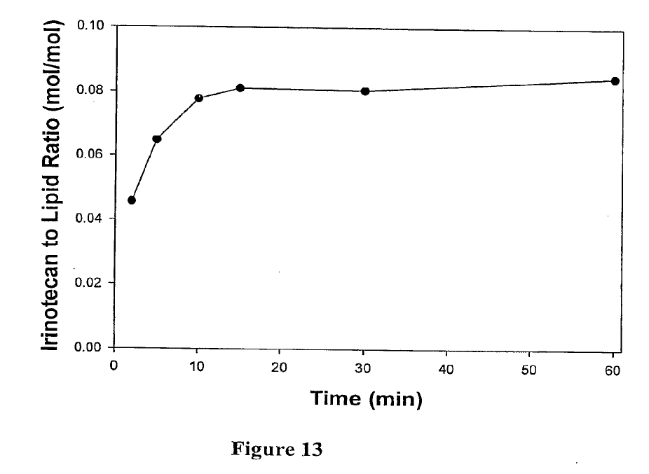

Using a diethanolamine (DEA) buffered internal solution at pH 7.0 and an external sucrose/phosphate/EDTA buffer, irinotecan was successfully encapsulated by the liposomes. (FIG. 13).

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the formulations and/or methods of use of the invention, may be made without departing from the spirit and scope thereof. U.S. patents and publications referenced herein are incorporated by reference.

1. A method of preparing a liposomal composition of at least one therapeutic agent, the method comprising:
i) providing a liposomal composition comprising a mixture of liposomes in an aqueous solution, wherein said liposomes have an internal solution comprising a secondary or tertiary amine aqueous solution, wherein said internal solution is buffered at a neutral pH;
ii) adding a first therapeutic agent to an external aqueous solution, wherein said external solution is a pharmaceutically acceptable buffer lacking a secondary or tertiary amine and buffered at a neutral pH, and wherein said first therapeutic agent has a protonatable amino group;
iii) maintaining said agent in the external solution for sufficient time to cause encapsulation of said agent into said liposomes.

2. The method of claim 1, wherein said secondary or tertiary amine aqueous solution in said internal solution is a secondary or tertiary alkylamine aqueous solution.

3. The method of claim 2, wherein said secondary or tertiary alkylamine is an alkanolamine.

4. The method of claim 3, wherein said alkanolamine is diethanolamine or triethanolamine.

5. The method of claim 1, wherein said internal solution further comprises a transition metal ion.

6. The method of claim 5, wherein said transition metal is copper.

7. The method of claim 6, wherein said copper is provided in a copper gluconate solution.

8. The method of claim 1, wherein said internal solution further comprises a sodium gluconate solution or a gluconic acid solution.

9. The method of claim 1, wherein said internal solution further comprises a phosphate or hydrochloric acid solution.

10. The method of claim 1, wherein said pharmaceutically acceptable buffer is a phosphate buffer.

11. The method of claim 1, wherein said first therapeutic agent is a anthracycline, a campthothecin, or a vinca alkaloid.

12. The method of claim 1, wherein said first therapeutic agent is doxorubicin, daunorubicin, irinotecan, topotecan, vincristine or vinblastine.

13. The method of claim 1, wherein at least one second therapeutic agent is added to said external solution simultaneously with said first therapeutic agent.

14. The method of claim 1, wherein at least one second therapeutic agent is added to said external solution sequentially relative to said first therapeutic agent.

15. The method of claim 1, wherein said liposomes are a mixture of 1,2-distearoyl-sn-glycero-3-phosphocholine (DSPC), 1,2-distearoyl-sn-glycero-3-phosphoglycerol sodium salt (DSPG), and cholesterol.

16. The method of claim 15, wherein said mixture of DSPC, DSPG and cholesterol is in a molar ratio of 7:2:1.

17. A liposomal composition prepared by the method of claim 1.

18. A liposomal composition comprising at least one therapeutic agent having a protonatable amino group; and a neutrally buffered secondary or tertiary amine.

19. The liposomal composition of claim 18, wherein said secondary or tertiary amine is a secondary or tertiary alkylamine.

20. The liposomal composition of claim 19, wherein said secondary or tertiary alkylamine is an alkanolamine.

21. The liposomal composition of claim 20, wherein said alkanolamine is diethanolamine or triethanolamine.

22. The liposomal composition of claim 18, wherein said therapeutic agent is irinotecan and said neutrally buffered tertiary amine is triethanolamine.

23. The liposomal composition of claim 22, further comprising copper gluconate.

24. The liposomal composition of claim 22, further comprising sodium gluconate.

25. The liposomal composition of claim 18, wherein the liposomes are a mixture 1,2-distearoyl-sn-glycero-3-phosphocholine (DSPC), 1,2-distearoyl-sn-glycero-3-phosphoglycerol sodium salt (DSPG), and cholesterol.
